(19) United States
(12) Patent Application Publication
(10) Pub. No.: US 2010/0226982 A1
(43) Pub. Date: Sep. 9, 2010

(54) Stabilized Active Ingredient Composition

(75) Inventor: Ralf Malessa, Essen (DE)

Correspondence Address: RANKIN, HILL & CLARK LLP, 23755 Lorain Road - Suite 200, North Olmsted, OH, 44070-2224, US

(73) Assignee: DR. SUWELACK SKIN & HEALTH CARE AG, Billerbeck (DE)

(21) Appl. No.: 12/377,613

(22) PCT Filed: Aug. 16, 2007
(86) PCT No.: PCT/EP07/58543

§ 371 Date: May 21, 2009

Related U.S. Application Data
(63) Continuation-in-part of application No. PCT/EP2007/058543, filed on Aug. 16, 2007.

(30) Foreign Application Priority Data
Aug. 17, 2006 (DE) 10 2006 038 629.9

(51) Int. Cl. A61K 9/10; A61K 31/375; A61K 8/67; A61Q 5/00; A61P 43/00

(52) U.S. Cl. 424/484; 514/474; 424/70.1

(57) ABSTRACT

The invention relates to a stabilized active ingredient composition comprising at least one carrier material and at least one active ingredient present in stabilized form, and at least one agent for forming the active ingredient from the stabilized form upon addition of an aqueous phase to the composition, wherein the composition is obtained by freeze drying, a process for producing the composition as well as the use of the composition.

The present invention relates to a composition comprising at least one carrier material and at least one active ingredient present in stabilized form, and at least one agent for forming an active ingredient from the stabilized form upon addition of an aqueous phase to the composition, wherein the composition is obtained by freeze drying, processes for its production as well as its use as a cosmetic or therapeutic agent, in particular for external application.

A number of important and potent active ingredients for external application in cosmetic or pharmaceutical agents are known for being instable and for being altered or degraded due to external influences in such a way that they can no longer comply with the desired action in the composition containing them, or can no longer do so to a sufficient extent, or that the altered or degraded products even display a harmful action. This applies particularly to thermolabile, light-sensitive, moisture-sensitive and/or oxidation-sensitive substances, but also to highly volatile fragrant substances. However, in order to be able to ensure and offer particular actions and effects, in particular in the case of cosmetic agents, and to thus prevail in a market with constantly growing consumer expectations with respect to the quality and efficacy of the products, there is a central interest in being able to make such instable ingredients available in the long term and efficiently, and highly active in application, in particular in aqueous and/or water-containing formulations.

Numerous processes were developed to stabilize such instable, readily decomposing and/or highly volatile substances and to make them available, also in the long term, in cosmetic and/or pharmaceutical compositions.

Thus, various processes for encapsulating active ingredients are known from the prior art, such as, for example, the use of liposomes or microspheres, these methods being particularly suitable for introducing instable active ingredients into liquid or semisolid formulations, such as creams, gels, lotions etc.

Thus, EP 0120722, for example, describes the use of liposomes for encapsulating instable and/or hydrophobic active ingredients. The use of micro capsules for stabilizing active ingredients is described, for example, in US2002/064541.

Apart from the use of such encapsulation systems, an active ingredient stabilization in the form of special emulsion techniques is known, such as in U.S. Pat. No. 6,171,600, or the stabilization with particular solvents is described, such as in WO 00/78283 or U.S. Pat. No. 6,103,267.

The difficult to doubtful re-release or reactivation of the active ingredients from the encapsulation is disadvantageous in the system of active ingredient encapsulation. Experts call into question the penetration of the liposomes or microspheres into the skin (J. W. Wiechers, Cosmetics & Toiletries Magazine Vol. 120 No. 6, 2005).

It is more probable that the encapsulation is dissolved on the surface of the skin. In the process, the encapsulation is broken up immediately prior to or during the application, and must primarily be ascribed to the mechanical influences, for example caused by applying the formulation onto, or rubbing/massaging the formulation into, the skin. A dissolution of the shell of the capsule by the skin's own enzymes, the skin's own pH value or other chemical influences is also conceivable. However, it is obvious that such a release of active ingredients can only be unspecific, and that a constant, reproducible dosing or application of the appropriate quantity of active ingredients is not ensured in these ways.

Stabilization by emulsion techniques and special solvents also involves certain drawbacks. For example, certain instable groups of active ingredients cannot be stabilized sufficiently and, at the same time, kept available with these systems. It is thus almost not possible to bring certain hydrophilic substances into solution in hydrophobic systems, such as, for example, vitamin C, which is extremely instable in aqueous systems; thus the useful use of emulsion techniques is not applicable in the case of such active ingredients. The use of hydrophilic non-polar solvents as described in WO 00/78283 is undesirable because additional chemical substances, which always also entail a certain toxicological or irritative potential, are thereby introduced into a dermatological formulation. In a time when the human body is increasingly exposed to external harmful influences and environmental stress, which is surely evident in the significantly increasing number of allergies, skin irritations and skin diseases, there is a clear trend towards formulations that are as natural as possible, in which the extent to which chemical additives are added should be kept as small as possible.

That is the reason why the stabilization of active ingredients, such as certain enzymes, by cross-linking with polymer systems is not desired. Such systems are described, for example, in JP 11246894 or JP 08038175 and by Kilinç et al, Turk J Chem 26, 311-316 (2002). Undesirable chemical derivatization reagents such as, for example, glutaraldehyde, the uncross-linked residues of which may remain in the product and lead to undesired skin reactions, are also being used in this case. Furthermore, the reaction products obtained by such a cross-linking process are often only unspecifically derivatized.

A significantly simpler and more specific process for providing such instable active ingredients which are light-labile, thermally labile and moisture-labil can be achieved, by introducing not the active ingredient itself, but a stable derivative form or a chemical prestage of an active ingredient, a so-called precursor, into the formulation. In many instances, the conversion of such derivatives of active ingredients and precursors may occur by simple chemical reactions, for example by a simple enzymatic reaction. The enzymes required for this are often even a natural component of the human skin. Thus, various documents can be found in which cosmetic or pharmaceutical compositions are described that contain active ingredients stabilized by derivatization or so-called precursors of active ingredients. Upon application onto the skin, the derivatives or precursors of active ingredients are converted into the active form or the actual active ingredient by the skin's own naturally present enzymes, so that this actual active ingredient is then released onto the skin. Examples of such a form of application can be found in DE 69503179, DE 69507517, DE 69500048 and in U.S. Pat. No. 6,569,906. This form of application, however, also has the decisive drawback of unspecific release and, furthermore, a very slow and oftentimes insufficient active ingredient release because of the generally relatively low enzyme activity on the skin.

In order to increase release kinetics, the enzymes releasing the active ingredient can already be added to the formulation in addition to the precursor of the active ingredient. This, however, gives rise to the big problem of preventing these two components from reacting already in the composition, thus releasing the active ingredient in the mixture already during or shortly after production. Thus, the instable form of the active ingredient would once again be contained in the composition, and stability and long-term availability of the active ingredients in the composition could not be guaranteed.

A widespread option of working around this problem is the provision of a precursor of the active ingredients and of the activator agent in a spatially separated arrangement. The spatial separation can in this case be effected by encapsulation of the reactive components or by the arrangement in a packaging having two compartments, which enables the combination no sooner than immediately prior to or during application. Such two-compartment systems are described, for example, in U.S. Pat. No. 5,788,972, US 2002/165271, FR 2855049, DE 69909563, DE 69520406 or also in WO 2004/058210.

The drawbacks of the encapsulation of active ingredients were already shown in detail; however, the use of packaging systems having different compartments or several chambers also entails obvious problems. Such systems are complex with respect to their production. Most of the time, conventional, commercially available standard packaging can not be used, but a costly new development with comprehensive tests, for example, for product compatibility, stability and/or safety becomes necessary.

Another form of instability of active ingredients is their volatility. There is thus the problem, in particular in highly volatile fragrant substances, that they evaporate and are thus not present in a sufficient concentration in the product used by the consumer, which of course also is a waste of these precious substances.

JP-A-09-187398 discloses so-called “tissue paper” (paper towels) having a retarded release of fragrant substances. For this purpose, the tissues are treated with a derivative of the fragrant substance. The tissues release the fragrant substance in a retarded manner under the influence of air humidity and microorganisms present in the air which transform the derivative of the fragrant substance into the fragrant substance. Enzymes which accelerate the release of the fragrant substances may also be added to the tissues. The application of the derivative of the fragrant substance and, optionally, of the enzyme, is carried out by spraying on an aqueous solution and air-drying the tissues, which immediately start the release of the fragrant substance in this manner. A release that would only begin with the use by the consumer (switch function) is not possible in this way. Furthermore, the process cannot be used for the production of freeze-dried compositions having a homogenous distribution of active ingredients. An administration of cosmetic or pharmaceutical active ingredients is not described. Such an administration would not be possible due to the intentional contamination with microorganisms during application. Similarly, JP-A-08-188525 by the same applicant describes the use in massage preparations of a derivative of an fragrant substance and of an enzyme releasing the fragrant substance upon addition of moisture. The application is cumbersome. The constituents mentioned can either be added to a dry massaging powder only, which entails difficulties in homogenizing the powders, or they must be added to water-containing massaging preparations, such as creams etc. immediately prior to application, which entails considerable problems with respect to dosing by the consumer. Neither document discloses the use of physiologically active cosmetic or pharmaceutical ingredients, nor the production of freeze-dried compositions.

Therefore, the object of the present invention consisted of providing a composition in which instable active ingredients can be kept stabilized in the long term and can be applied quickly, efficiently, specifically and highly actively during use, with stabilization preferably being achieved by the use of derivatized active ingredients and/or precursors of active ingredients, and wherein the quick, efficient and specific release or reactivation of the active ingredients is effected by suitable releasing agents, which are also contained in the composition, upon addition of an aqueous phase to the composition and without the necessity of keeping the active ingredients and the releasing agents apart from each other in the composition by complex encapsulation processes, chemical stabilization/cross-linking processes and/or packaging processes.

The inventors of the present invention found that the above-described problems of the prior art can be resolved by incorporating a stabilized form of the active ingredient together with an agent for forming the active ingredient in its non-stabilized form (hereinafter sometimes referred to as “releasing agent”) into a matrix of a carrier material by means of freeze-drying. This process permits the effective spatial separation of the stabilized active ingredient from the releasing agent in the carrier material and the prevention of a premature reaction of the substances with each other. By simple addition of an aqueous phase (preferably by the end consumer) the mobility of the stabilized active ingredient and of the releasing agent is then reestablished and the non-stabilized active ingredient is formed in a pure, i.e. highly active, form. Furthermore, the release rate of the active ingredient during application can additionally be controlled through the selection of the carrier material and the concentrations of the active ingredient and/or the releasing agent. Moreover, the process of the invention also permits the stabilization of highly volatile active ingredients, such as, in particular, fragrant substances, with respect to evaporation. This permits stabilization even of highly volatile active ingredients produced by freeze-drying, such as, for example, freeze-dried compositions for cosmetic application, which was not possible until now.

The composition according to the invention avoids the drawbacks of the known processes, it requires, in particular, no encapsulation of the active ingredients and no chemical stabilization or cross-linking agents or a complex packaging system or application system.

The invention thus provides a composition into which the instable active ingredients are incorporated in a stabilized form, e.g. as a derivative of active ingredients or as precursors or prestages of active ingredients. Release or reactivation of the actual active ingredient takes place upon or immediately prior to the application by means of releasing agents also incorporated into the composition. According to the invention, the premature reaction of these two groups of substances is prevented by incorporation into a suitable carrier material, preferably comprising a hydrocolloid, and subsequent freeze-drying, instead of by encapsulation, chemical stabilization or cross-linking or by complex packaging or administration systems. It is only upon addition of an aqueous phase to the composition that the substances immobilized in the carrier material are remobilized and come into contact with one another, whereby the reaction of the releasing agent with the stabilized active ingredient is enabled quickly, efficiently and completely.

Accordingly, the composition according to the invention comprises at least one carrier material and at least one active ingredient present in stabilized form, and at least one agent for forming an active ingredient from the stabilized form upon addition of an aqueous phase to the composition, with the composition being obtained by freeze drying.

The carrier material preferably is a hydrophilic material, i.e. a material that is wettable with water. Preferably, it is a so-called hydrocolloid, that is, a partly water-soluble natural or synthetic polymer which forms gels or viscous solutions in aqueous systems.

The hydrocolloids known from WO 2004/035023, WO 2004/104076 and DE 4028622 constitute, for example, the carrier material used according to the invention, i.e. (partially) water-soluble or water-swellable natural or synthetic polymers that form gels or viscous solutions in aqueous systems.

The carrier materials are expediently selected from the group of polysaccharides, glucosaminoglycanes, proteins and/or synthetic polymers. Preferably, the carrier material is selected from the group of polysaccharides. Polysaccharides include, for example, homoglycanes or heteroglycanes, such as, for instance, alginates, in particular sodium alginate, carrageen, pectins, gum tragacanth, guar gum, carob gum, agar-agar, gum arabic, xanthan gum, natural and modified starches, dextrans, dextrin, maltodextrins, chitosan, glucans, such as β-1,3-glucan or β-1,4-glucan, cellulose etc.

Glucosaminoglycanes (mucopolysaccharides) include, for example: hyaluronic acid, chondroitin sulfate, dermatan sufalte, keratan sulfate, heparan sulfate, heparin, etc.

Hydrocolloid-forming proteins include e.g. collagen, gelatin, elastin, keratin, fibroin, albumins, globulines such as lactoglobulin, milk proteins such as casein etc.

Synthetic polymers include, for example: cellulose ether, polyvinyl alcohol, polyvinyl pyrrolidone, synthetic cellulose derivatives, such as methylcellulose, carboxycellulose, carboxymethylcellulose, cellulose ester, cellulose ether such as hydroxypropylcellulose, polyacrylic acid, polymethacrylic acid, poly(methylmethacrylate) (PMMA), polymethacrylate (PMA); polyethylen glycols, etc.

Mixtures of several carrier materials can also be used.

Particularly preferred polysaccharides are alginates, sodium alginates are particularly preferred, in particular calcium-free sodium alginates (sodium alginates with a calcium content of less than 3% by wt., more preferably less than 2% by wt., still more preferred less than 1.5% by wt.). Such alginates are particularly preferred that have a viscosity of less than 2000 mPas, more preferably of less than 1000 mPas, most preferably less than 100 mPas (i.e., a solution of 1 g of the carrier material in 99 ml distilled water (1% by wt. solution) at 20° C. and a pH value of 6 to 8 has a viscosity of less than 2000, 1000 or 100 mPas, respectively (Haake VT 500 viscometer, shear rate 50 l/s, measurement body MV 1).

The use of such carrier materials, such as calcium-free sodium alginates, is, on the one hand, preferred with respect to the production process, on the other hand, quickly soluble compositions according to the invention can be obtained by using such carrier materials. A ready solubility of the formulation according to the invention, or a high rate of disintegration or dissolution upon addition of water or aqueous solutions leads, among other things, to a better dispersability on the skin and is desired according to the invention. In particular the use of low-viscosity alginate types can lead to a higher dissolution rate of the molded articles used according to the invention. Furthermore, this is of particular significance in order to ensure a quick remobilization of the active ingredient system and the releasing agent and thus, an as immediate and complete reaction of the two substance groups with each other as possible, which in turn is essential for an optimal release of the active ingredient and thus, for an availability of the reactivated active ingredient in the composition that is as good and quick as possible.

The dissolution rate of a readily soluble composition according to the invention, measured in accordance with a method for measuring the “disintegration rate of tablets and capsules” with a testing apparatus according to PharmEU, preferably is less than 4 minutes, more preferably less than 1 minute (in the case of molded articles having a diameter of 9 mm, complete hydration without an observable core is present after <20 seconds).

The carrier materials preferably used in the composition according to the invention are polysaccharides that have average molar masses of, expediently, approximately 10³ to about 10⁸, preferably of about 10⁴ to 10⁷.

For another preferred embodiment of the invention, the active ingredient, which is present in a stabilized form, and at least one agent for forming the active ingredient from the stabilized form upon addition of an aqueous phase to the composition are incorporated into a carrier material as it is known from WO 2004/104076 and WO 2005/113656. In the process, the carrier material from alginates of multivalent metal ions is cross-linked with salts of multivalent metal ions, whereby insoluble, swellable freeze-dried preparations according to the invention are obtained, which can be used, in particular, as masks or packs, and which, when applied onto an appropriate part of the body, locally release the highly active ingredients.

Also preferred is an embodiment of the invention which results when collagen is used as a carrier material. Collagen is a protein which belongs to the class of hydrocolloids. Collagen processed in accordance with processes known from the prior art, and, for example, from DE 4028622, is preferably used. This collagen carrier material is characterized in particular by its excellent hydration properties and by the particularly good tolerability due to structural similarity to the human skin, and it is therefore, according to the invention, particularly suitable as a carrier material for stabilized active ingredients for external application.

The compositions according to the invention contain at least one or several stabilized and/or inactivated active ingredients admixed to the carrier material. Active ingredients include, in particular, cosmetic or therapeutic or pharmaceutical active ingredients suitable for external application. Preferably, the carrier material used according to the invention contains at least one cosmetic and/or pharmaceutical active ingredient. Accordingly, the composition according to the invention preferably is a cosmetic or pharmaceutical product. Cosmetic compositions or compositions prepared using cosmetic active ingredients within the meaning of the invention essentially are products within the meaning of the “Lebensmittel-, Bedarfsgegenstande- and Futtermittelgesetz” (German Food Law), i.e. substances and compositions exclusively or mainly intended for external use on the human body, the human oral cavity for cleaning purposes, for protection, for changing the appearance or for influencing body odor. Substances or compositions of substances intended for influencing body shapes are not deemed cosmetic reparations.

In this sense, the cosmetic compositions used according to the invention are, for example, bathing preparations, skin washing and cleansing products, skin care products, in particular face care products, in particular natural and synthetic moisturizing factors, eye cosmetics, lip care products, nail care products, foot care products, hair care products, in particular hair washing products, hair conditioning products, hair softening rinse etc., skin protection products, in particular antioxidants or light protection products, anti-irritative products, so-called anti-aging products, suntan products, skin lightening products, depigmentation products, deodorants, antihydrotics, depilatory products, insect repellents etc. or a combination of such products.

Examples of cosmetically, or optionally dermatologically therapeutically effective substances can be: anti-acne products, antimicrobial products, antitranspiration products, astringent products, deodorizing products, depilatory products, conditioning products for the skin, skin-smoothing products, products for increasing skin hydration, sun blockers, keratolytic products, free-radical scavengers for free radicals, antiseborrhoeic products, anti dandruff products, antiseptic active ingredients, active ingredients for treating signs of the aging of the skin and/or products modulating the differentiation and/or proliferation and/or pigmentation of the skin (e.g., melanin precursors), vitamins, active ingredients with a stimulating side effect, hydrating products and/or skin-soothing products.

Moreover, plant ingredient extracts or extracts obtained from them or individual substances can be mentioned. Generally, the active plant ingredient extract is selected from the group consisting of solid plant extracts, liquid plant extracts, hydrophilic plant extracts, lipophilic plant extracts, individual plant constituents and mixtures thereof.

In particular, cosmetic active ingredients within the sense of the invention do not include fragrant substances.

In contrast to the above described compositions which are substantially used in cosmetics, the therapeutically used compositions (medicaments) are such compositions containing at least one pharmaceutical or therapeutic, in particular dermatological, active substance, and which, within the meaning of the “Arzneimittelgesetz” (German Medical Preparations Act), are intended to cure, to ease or to prevent diseases, illnesses, bodily damage or pathological complaints. Such products or active ingredients are intended for external application, and they can be skin-active but also transdermal active ingredients. They include, for example: products for treating skin diseases, externally applicable analgetics, antirheumatics/antiphlogistics (NSAR), oxicams; steroid hormones, antigout agents, dermatic products, externally applicable products, including antibacterial products, antimycotics, antiviral active ingredients, anti-inflammatory active ingredients, antipruritic active ingredients, anesthetizing active ingredients, anti-acne products, antiparasitic active ingredients, externally applicable hormones, vein therapeutic products, immunosuppressives etc, all for external application.

Preferred therapeutic products are analgetics, e.g. immunosuppressives, hormones, products for the treatment of skin diseases such as neurodermatitis, atopical dermatitis, acne, rosacea etc., and anti-herpes products.

According to the invention, in particular such active ingredients from the classes of active ingredients are used in the present composition which cannot be incorporated with sufficient stability into conventional cosmetic or pharmaceutical compositions on an aqueous and/or fat-containing/oil-containing basis, such as creams, salves, lotions, gels, foams, sprays etc., owing to their high light lability, temperature lability, oxidation lability and/or moisture lability.

In the process, the active ingredient is incorporated in a stabilized form. According to the invention, an active ingredient which is present in a stabilized form in particular means that, under the same conditions, the concentration of the stabilized active ingredient is always higher on the time axis than the concentration of the active ingredient in the non-stabilized form. In other words, in a diagram in which the concentration of the non-stabilized active ingredient and the concentration of the active ingredient in its stabilized form are plotted over time, the curve for the active ingredient in its stabilized form is always above the curve for the non-stabilized active ingredient, that is, the active ingredient which is present in its free form, in a manner of speaking, which therefore decomposes more quickly. According to the invention, this stabilization may in principle take place in various different ways. For example, derivatives of active ingredients can be used, which, by definition, are the progeny of a compound which can be formally derived from a basic compound or be produced from it (Fachlexikon ABC Chemie, 3rd edition, 1987, Harri Deutsch publishing company).

However, prestages of active ingredients, so-called precursors of active ingredients, can also be used.

Both the derivatives as well as the precursors naturally must have a higher stability than the actual active ingredient. Moreover, the active ingredient must be released from the stabilized form upon addition of an aqueous phase, preferably without any byproducts being produced which have, in particular, toxic, allergizing, irritating or similar effects, or other undesirable negative properties, such as an unpleasant smell of its own, undesirable color development or similar properties.

The precursor or derivative of the active ingredients itself should also be physiologically tolerable and have, in particular, a good cutaneous tolerability and be free of the above described undesirable properties.

Upon addition of the aqueous phase, and after an optional homogenization at 20° C., preferably at least 5% by wt., more preferably at least 10% by wt. of the free non-stabilized active ingredient, relative to the total amount of the stabilized active ingredient present, are released, preferably within 60 seconds, more preferably within 30 seconds. The releasing conditions may of course vary for a given system of stabilized active ingredient and releasing agent.

For example, derivatives or precursors of vitamins, ketoses (e.g., dihydroacetone/DHA), mono- and/or diesters of cinnamic acid or derivatives thereof, derivatives of hydroxy acids (e.g., lactic acid, glycolic acid, malic acid, tartaric acid, citric acid, 2-hydroxalcanoic acid, mandelic acid, salicylic acid), derivatives of quercetin, nucleotide precursors, phosphate-containing hydroxyacetones, glycerin precursors etc. may be used.

Generally, amides and/or sugar derivatives of active ingredients can be used as derivatives and precursors. Derivatives of glucose, mannose, ribose, fructose, fucose, N-acetylglucosamine and/or N-acetylgalactosamine as well as derivatives of N-acetylmuramic acic and/or derivatives of sialic acid and/or mixtures thereof are possibilities from the group of sugar derivatives. Peptides such as lipotyrosine and/or trityrosine are possibilities from the group of the amide derivatives.

Also relevant, according to the invention, is the use of ester and/or ester derivatives. In particular, esters from reactions with inorganic acids, such as phosphate or sulfate esters, and alkyl and/or acylesters from reactions with organic acids such as lauric acid, myristic acid, palmitic acid, stearic acid, cetylic acid, linoleic acid, linolenic acid, octanoic acid, oleic acid and/or acetic acid, propionic acid and/or butyric acid and/or with hydroxy acids such as glycolic acid, lactic acid, tartaric acid, citric acid, salicylic acid and/or ricinoleic acid or also with, cinnamic acid play a role in the process. Furthermore, esters of fatty alcohols, such as dodecyl, hexadecyl, stearyl, cetyl, myristidyl, linoleyl, octyl, and/or oleyl alcohol, as well as esters of butyl, propyl and/or ethyl alcohol and also esters of polyols, such as propylene, butylene glycol and/or glycerin and mixtures thereof can be used according to the invention.

Possible derivatives of DHA are, for example dihydroxyacetone monolaurate, -dilaurate, -monostearate, -distearate, -monopalmitate and/or -dipalmitate. Gylcerin trilactate, ethyl lactate and sulfate-containing derivatives can be specified as possible derivatives of the lactic acid. Glycerin trilactate and (3-glycerophosphates, which release glycerin as an active ingredient, must be mentioned as examples for glycerin derivatives. Quercitin glucoside and/or quercitin ester must be mentioned from the group of the quercitin precursors. Examples of nucleotide precursors are adenosine phosphate, guanosine phosphate, cytosine phosphate, uridine phosphate, thymidine phosphate, inosine phosphate as well as xanthosine phosphate.

Derivatives or precursors of active ingredients from the group of vitamins and vitamin derivatives, such as, for example, vitamin A (retinoids such as retinol, retinal, retinic acid), vitamin B, ascorbic acid (vitamin C), vitamin D, tocopherols (e.g. vitamin E), vitamin F etc. are preferably used. In particular esterified vitamin derivatives such as retinyl palmitate, propionate, acetate, butyrate, octanoate, laurate, oleate and/or linoleate, or also tocopherol esters such as tocopherol nicotinate and/or acetate as well as phosphates, sulfates, palmitates, acetates, nicotinates and/or propionates of the vitamins A, C and/or E as well as sugar derivatives of these vitamins are used in the process. Phosphates such as those from alkaline, alkaline earth and/or transition metals, such as magnesium, sodium, potassium, calcium and/or zinc can be used.

Particularly preferably used vitamin derivatives are those from ascorbic acid (vitamin C), a water-soluble vitamin, which is highly at risk of being oxidized, in particular in the presence of heavy metal traces (e.g. copper and iron), but also under the influence of light and/or alkaline, and which is of great significance as a cosmetic and therapeutic active ingredient. Preferably used ascorbic acid derivatives are ascorbic acid esters such as ascorbyl palmitate, laurate, myristate, stearate and/or nicotinate; magnesium ascorbyl phosphate is particularly preferably used, since that ester of the ascorbic acid is significantly more stable as compared with the light and oxidation-sensitive vitamin C. Other possible ascorbic acid derivatives are those from reactions of ascorbic acid with sugars such as glucose, mannose, fructose, N-acetylglucosamine, fucose, galactose, N-acetylgalactosamin, sialic acid and/or N-acetylmuramic acid and mixtures thereof.

According to the invention, the stabilized active ingredient can also be selected from the group of fragrant substance precursors or fragrant substance derivatives. Fragrant substances and aromas are characterized, in particular, by being highly volatile and thus exceptionally temperature-sensitive. This makes processing fragrant substances or aromas in compositions that are supposed to have a long-term stability, such as, for example, cosmetic and/or pharmaceutical compositions, difficult in cases where these compositions are to be subjected to a temperature treatment.

In particular; this also applies to cosmetic and/or pharmaceutical compositions that are subjected to a freeze-drying process. In the freeze-drying process, water is extracted by sublimation from, for example, an aqueous or water-containing formulation, by freezing and subsequent reduction of pressure already at temperatures significantly below the boiling point of water. It is obvious that, under these conditions, all other contents having boiling points below that of water are also removed from the formulation, such as, for example, alcohols as well as particularly highly volatile fragrant and aromatic substances.

The principle according to the invention of stabilization and re-releasing of instable active ingredients can thus also be applied to the group of the fragrant and aromatic substances. For the first time, in addition to the possibility of stabilizing fragrant substances, this also yields the possibility of producing freeze-dried cosmetic products with fragrant and aromatic substances. This is of interest inasmuch as fragrant substances play a central role, in particular in the cosmetics industry. Pleasant scents can trigger numerous pleasant feelings or associations in the user and thus affect the experience of using them in a positive manner, increase the pleasure of using them and thus increase product acceptance in a sustained manner.

The composition according to the invention, in accordance with the principle presented in detail above, is a composition in which, upon addition of an aqueous phase to the composition, the derivatives and/or precursors of the fragrant substances immobilized in the carrier material and the substances for forming the active fragrant ingredient are remobilized from the stabilized form. Thereby, the reaction of the releasing agent with the stabilized active ingredient is enabled quickly, efficiently and completely, also in this group of substances, and the composition displays the desired effect, the pleasant scent.

Generally, the above listed groups such as amides and/or ose derivatives (sugar derivatives) ester and/or ether derivatives, can be used as derivatives and precursors also in this case.

Menthlyllactate is particularly preferably used as a precursor/derivative of an fragrant substance.

According to the invention, the immobilized derivatives and/or precursors of the fragrant substances preferably are esterified derivatives of fragrant substances such as, for example, esters of perfume alcohols. These may be completely esterified perfume alcohols or perfume alcohol esters having one or more free carboxylate groups or also mixtures thereof. Examples of such perfume alcohol esters are described in detail, for example, in U.S. Pat. No. 5,721,202.

Geraniol, nerol, phenoxanol, floralol, β-citronellol, nonadol, cyclohexyl, ethanol, phenylethanol, phenoxyethanol, isoborneol, fenchol, isocyclogeraniol, 2-phenyl-1-propanol, 3,7-dimethyl-1-octanol, for example, can be taken into consideration from the group of perfume alcohols.

Maleates, succinatadipates, phthalates, citrates or also pyromelliate esters of the perfume alcohols may be selected as esters.

Geranylsuccinate, nerylsuccinate, β-citronellylmaleate, nonadolmaleate, phenoxanolmaleate, (3,7-dimethyl-1-octanyl)succinate, (cyclohexylethyl)maleate, floralylsuccinate, (β-citronellyl)phthalates and/or (phenylethyl)adipates may be mentioned from the group of perfume alcohol esters having free carboxylate groups.

Digeranyl succinate, dineryl succinate, geranylneryl succinate, geranylphenyl acetate, nerylphenyl acetate, geranyl laurate, neryl laurate, di(β-citronellyl)maleate, dinonadol maleate, diphenoxanyl maleate, di(3,7-dimethyl-1-octanyl)succinate, di(cyclohexylethyl)maleate, difloralyl succinat and/or di(phenylethyl)adipate may be mentioned, for example, from the group of completely esterified perfume alcohols.

The derivatives and/or precursors of active ingredients used according to the invention possess a significantly better stability with respect to external influences, such as moisture, oxidation, temperature, etc. However, in order to rerelease the actually active constituent upon addition of an aqueous phase in a quick, efficient and highly active manner, so-called releasing agents or activators are required. In the process, the releasing agents enter a chemical reaction with the derivative and/or precursor, and the actual active ingredient is, for example, split off, possible stabilizing protective groups are separated, or the actual active ingredient is produced directly by a conversion reaction.

A catalytic effect of the releasing agent is also possible.

Oxidants/reductants, catalysts, such as, in particular, enzymes, metal-catalyzed systems or substances that alter the pH value, such as acids or bases, can thus be used as releasing agents.

Another pH altering substance, which can be used as a releasing agent according to the invention, is γ-butyro-δ-lactone, for example.

According to the invention, enzymes are particularly preferably used in order to form the active ingredients from the stabilized derivatives or precursor substances. From the group of the enzymes, those from the sub-group of the oxidoreductases (enzymes of biological oxidation and reduction), such as, e.g., dehydrogenases, oxidases, peroxidases, dioxygenases or monoxigenases, from the group of the transferases (group-transferring enzymes), such as, e.g., transferases, synthases or transmainases, and/or from the group of the hydrolases (enzymes catalyzing hydrolytic splits), such as, e.g., lipases, phospatases, amylases, peptidases, esterases or proteases are preferably used.

The aqueous phase under whose influence the releasing agent releases the active ingredient from the stabilized form may contain, in particular, pure water, such as normal spring water or tap water, or specially produced aqueous compositions, which may also contain solvents, such as alcohols, and optionally also other constituents, such as the auxiliary substances mentioned below. The aqueous phase preferably contains more than 70, more preferably more than 80, still more preferably more than 90% by wt. of water.

The composition according to the invention furthermore optionally contains one or more auxiliary substances. Auxiliary substance incude, for example: fatty substances such as mineral oils, paraffin oils or vaseline oils, silicon oils, refined or unrefined vegetable oils, vegetable lecithins (e.g. soy lecithin), sphingolipids/ceramids isolated from plants, animal oils or fats, fatty acid esters, esters of fatty alcohols and waxes having a melting point corresponding to skin temperature (animal waxes, mineral waxes and synthetic waxes), as well as all oils suitable for cosmetic purposes, such as mentioned, for example, in the CTFA treatise, Cosmetic Ingredient Handbook, 1st ed., 1988, The Cosmetic, Toiletry and Fragrance Association, Inc., Washington, polyunsaturated fatty acids, essential fatty acids, surface-active agents such as washing surfactants, detergents, foam-forming agents or dispersing agents, emulgators, etc. fillers, pH-regulating agents, such as buffering substances, stabilizers, cosolvents, pharmaceutically and cosmetically common or other colorants and pigments, preservatives, softening agents, lubricants or slip additive agents, etc. Particularly preferred auxiliary substances are selected from the group of fats, oils and waxes, with particularly preferred auxiliary agents being capryl/capric acid triglycerides, squalane and glycerin as well as shea butter.

The classification of the above-mentioned substances into the category of auxiliary substances within the context of the present invention does not preclude these auxiliary substances from also having certain cosmetic and/or therapeutic effects.

The composition according to the invention is preferably present as a molded article after the freeze-drying process. A molded article within the meaning of the invention is understood, in the present context, to be a regularly formed geometrical body, e.g., in particular, spheres, cuboids, pyramids, stars, but also molded articles copied from natural shapes, such as, e.g. those in the shape of animals, such as, e.g. marine animals, such as, e.g. starfishes, seafood, such as mussels, etc., plants and parts of plants, such as leaves, etc. In accordance with the process for producing the molded articles used according to the invention described below, all of these shapes are obtainable. According to the invention, a plurality of the molded articles mentioned is also included in a container. It may also be a mixture of molded articles of different geometries. The molded article may be packaged individually. Preferably, however, a plurality of the molded articles are present, side by side, in a container, in particular in cosmetic use.

The volumes of the molded articles used are not limited per se due to the production process. Expediently, the volumes are preferably at least approximately 0.1 cm³, preferably 0.3 cm³, still more preferably at least 0.5 cm³. The volumes used are expediently given an upper limit of up to about 6 cm³, preferably of up to 5 cm³, more preferably of up to 4 cm³. The size of the molded articles is determined, among other things, by the location of the external application of the molded articles. Thus, the application on larger body surfaces or on the hair (e.g, the direct application of the moistened molded articles onto the back, etc., or the use as a bathing preparation) enables the use of larger molded articles, whereas smaller molded articles are preferred where application on smaller body areas (e.g., the cheek, etc.) is concerned.

The diameter of a molded article (maximum distance between two points in a molded article of any geometry) expediently is at least about 3 mm, preferably at least about 5 mm, more preferably at least about 7 mm, still more preferably at least about 8 mm to up to, expediently, 60 mm, preferably about 50 mm, more preferably 40 mm, still more preferably about 30 mm.

A particularly preferred molded article has a substantially spherical geometry, with the diameter of the sphere being between 3 to 30 mm, preferably between 5 and 20 mm, still more preferably between 7 and 15 mm, even more preferably between 8 and 13 mm.

The composition according to the invention can, however, also be present as a sheet, layer, fleece, film, or granulates.

A particularly preferred composition according to the invention has one, several, or all of the following features:
it contains at least 10% by wt. of one or more carrier materials, whose 1% by wt. solution or suspension preferably has a viscosity of less than 2000, or 1000, or 100 mPas, respectively, in water at 20° C. and pH 6-8, which includes, in particular, alginates, with sodium alginates being preferred,
it contains 0.000001% by wt. to up to 50% by wt. of one or more active ingredients which are present in stabilized form, preferably in the form of derivatives and/or precursors of active ingredients, in particular selected from the group of vitamins and/or fragrant substances,
it contains 0.000001% by wt. to up to 50% by wt. of one or more agents for forming the active ingredient from the stabilized form upon addition of an aqueous phase to the composition, in particular selected from the group of enzymes,
it contains 0.1 to 70% by wt of one or more auxiliary substances, in particular from the group of fatty substances,
it preferably contains less than 10% by wt, more preferably less than 5% by wt, still more preferably less than 3% by wt of water,
   the weight in each case specified relative to the total composition.

In addition, the composition according to the invention, such as, for example, the above-mentioned composition containing at least one carrier materials, optionally one or more additional active ingredients present in stabilized form, as well as optionally one or more agents for forming the active ingredient from the stabilized form upon addition of an aqueous phase to the composition, as well as optionally one or more auxiliary substances, preferably has at least one of the following features:

the geometric form of a sphere or a sheet or of a layer or a fleece

a density of 0.005 g/cm³ to 0.8 g/cm³, preferably 0.01 g/cm³ to 0.8 g/cm³,

a volume of 0.1 cm³ to 6 cm³, preferably 0.5 cm³ to 6 cm³,

a diameter (maximum distance between two points of the molded article) of at least 6 mm, or

a thickness (shortest distance of two points, i.e. layer thickness) of 1 mm to 25 mm.

The composition according to the invention is obtainable by a process comprising the following steps:

(a) preparing a solution or suspension comprising at least one carrier material, one or more active ingredients in stabilized form, preferably in the form of derivatives and/or precursors of active ingredients, as well as at least one agent for forming the active ingredient from the stabilized form upon addition of an aqueous phase to the composition, as well as, optionally, one or more auxiliary substances
(b) pouring the solution or suspension into a mold,
(c) freezing the solution or suspension in the mold and
(d) freeze-drying the frozen solution or suspension while forming the freeze-dried composition or the freeze-dried molded article.

Optionally, other steps may be executed in between these steps; it is possible, in particular, in step (a), to set the pH value of the solution or suspension such that the admixed enzymes are inactive, or to add salts of multivalent metal ions for the purpose of cross-linking the carrier material. Following step (c) or (d), a processing of the surface of the frozen or freeze-dried composition can take place by mechanical processing or by spray-application of solutions of active ingredients, of colorants and/or agents modifying the dissolution rate. Preferably, the composition has no surface coating, however, and is composed homogeneously within the sense of an equal distribution of the constituents over the entire composition.

Expediently, production is effected by first producing an aqueous solution or suspension of the carrier materials and then adding and admixing within the shortest possible time one or more active ingredients in stabilized form, as well as at least one agent for forming the active ingredient from the stabilized form upon addition of an aqueous phase to the composition, as well as, optionally, one or more auxiliary substances. Processing takes place, in accordance with the temperature, preferably at <10° C., particularly preferably at <4° C. This temperature range is preferred because the enzymes that are preferably added exhibit an activity minimum here, which inhibits a premature reaction with the stabilized active ingredient in the aqueous solution or suspension of the composition from step (a). Expediently, the time for mixing the solution or suspension and the following process steps until freezing are kept as short as possible, preferably, this time span is less than 8 seconds, more preferably less than 3 seconds. The admixing of the stabilized active ingredient and the releasing agent into the aqueous solution or suspension of the carrier material preferably takes place by online admixing directly before the extrusion or the pouring or loading into the molded article molds.

Another option for inhibiting the premature reaction of the admixed substances can be obtained by adding volatile chemicals with an inhibiting effect as auxiliary substances to the solution or suspension. These inhibitory substances exercise their protective effect only in the state of the aqueous solution or suspension; these volatile inhibitors are removed during the freeze-drying process according to step (d). The inhibitor effect after the freeze-drying process then occurs because of the mechanism of spatial separation of the substances in the carrier material, which was already explained in detail.

In order to provide the freeze-dried composition with a sufficient mechanical stability, it is necessary for the solution or suspension to have a certain concentration of the carrier material. Of course, this concentration depends on the type of hydrocolloid used. Expediently, it is about at least 0.1% by wt. relative to the total quantity of the solution or suspension, preferably about 0.25% by wt. to about 20% by wt., more preferably less than 15% by wt., still more preferably less than 10% by wt. (weight of the carrier material relative to the total weight of the solution or suspension). Higher concentrations are not preferred, because in that case, the viscosity of the solution or suspension becomes too high, thus making the solution or suspension more difficult to process. The quantity of the carrier material contained in the solution or suspension has a decisive effect on the density of the composition obtained (weight of the composition relative to the volume of the geometric shape of the composition). The density, in turn, is an important quantity for the dissolution rate of the composition upon the addition of an aqueous phase or when wetted with water or a solution of an active ingredient and/or an auxiliary substance. The higher the concentration of, the carrier material in the solution or suspension, the higher the density (the lower the degree of porosity) of the composition becomes and vice versa. In view of the density/degree of porosity or dissolution rate, the concentration of the carrier material in the solution or suspension prepared in step (a) is preferably selected from a range of about 0.25% by wt. to about 15% by wt., relative to the solution or suspension. The concentration of the preferably used plant hydrocolloid sodium alginate is preferably from 0.5 to 5% by wt., more preferably 1 to 4% by wt.

A concentration of preferably 0.2 to 3% by wt. alginate is used for producing the preferred insoluble embodiment from cross-linked alginate.

The concentration of the also preferably used proteinogen hydrocolloid collagen preferably is from 0.5 to 5, more preferably from 1 to 3% by wt., relative to the solution or suspension.

Expediently, the densities of the compositions obtained in accordance with the invention are about 0.005 g/cm³ to 1.0 g/cm³, preferably about 0.01 g/cm³ to 0.5 g/cm³, preferably about 0.02 g/cm³ to 0.2 g/cm³. The term density as it is presently used denotes the weight of the composition relative to the volume of the exterior geometric shape of the composition.

The weight of the individual molded articles for which the composition according to the invention is formed is, of course, dependent upon their size. Generally, the weight of the individual molded articles is about 10 to 200 mg, preferably 20 to 150 mg. For example, spheres with a diameter of 12 mm have a weight in the range from, preferably, 20 to 250 mg, more preferably 30 to 200 mg. Corresponding preferred ranges are calculated for spheres having other diameters. In embodiments in the form of, for example, sheets, layers or fleeces, the length and width of the composition are at least ten times, preferably at least 20 times the size of the thickness, they can be cut or punched into shapes, and have surfaces of preferably at least about 25 cm², more preferably of at least about 50 cm², still more preferably of at least 100 cm².

Preparation of the Solution or Suspension that is Subjected to Freeze-Drying is preferably effected such that, first, a suitable solution or suspension of the carrier material is produced, and that, then, the active ingredients in a stabilized form, releasing agents for forming the active ingredient from the stabilized form upon addition of an aqueous phase to the composition and, optionally, auxiliary substances are incorporated into the solution or suspension. If oil-soluble derivatives/precursors of active ingredients are used, they are preferably dissolved in oils (in particular squalane, caprylic/capric triglycerides), which are optionally used as auxiliary substances, and are then added to the solution or suspension of the carrier material, which has the advantage that stable solutions or suspensions form. No emulsifiers are required, and no phase separation of the solution or suspension occurs during processing if oil-soluble or oily derivatives/precursors of auxiliary substances or active ingredients are used. It is also possible, however, to disperse the stabilized active ingredients in the solution or suspension.

The solution or suspension thus produced is then poured into a mold which has cavities of the desired geometric shapes corresponding to the molded articles to be produced. The mold preferably consists of rubber, silicone rubber, vulcanized rubber (rubber) etc. Rubber molds are preferred. The mold materials may optionally be coated. The cavities of the molded article molds into which the solution or suspension is poured generally have the shape of the desired molded article. That is, the volume of the cavity substantially corresponds to the volume of the molded article that is obtained later.

Since the volume of the solutions or suspensions increases in the cavities upon being frozen (difference in density between water and ice), the cavities are generally not filled completely. In this way, completely symmetrical molded articles are obtained.

After the solution has been filled into the cavities of the mold, the solution or suspension is frozen. The solution may, as such, cool off or freeze in the mold in an arbitrary manner. Preferably, the cooling-off in the method used according to the invention is effected by blowing cold air at it. Other methods include, for example, immersing the molds into liquid gases, such as, for example, immersion into liquid nitrogen. The cooling rate in the process has an effect upon the size of the ice crystals formed. They in turn have an effect upon the pore size distribution of the molded article formed. If few large crystals are formed, then the molded article has few large pores. If many small crystals are formed, then the molded article has many small pores. The higher the cooling-off rate of the solution or suspension, the smaller the crystals are.

The freezing temperature required depends, among other things, on how far the freezing point has been lowered by the derivatives/precursors of active ingredients, releasing agents or auxiliary substances contained in the solution or suspension. Expediently, the temperature is below the freezing point of water down to the temperature of liquid nitrogen (−196° C.). Preferably, the freezing temperature is about −20° C. to −80° C. After the solution or suspension has frozen, the molded articles are removed from the mold and subjected to subsequent processing, if necessary. Subsequent processing can take place mechanically, e.g., by surface processing (grinding, roughening).

The molded articles are then subjected to the freeze-drying process. Freeze drying can take place in a manner known per se, such as described, for example, in DE 4328329 C2 or DE 4028622 C2. As regards the process parameters, a drying temperature in the range from −20 to +100° C. in a vacuum of about 0.1 to 3.0 mbar is preferably selected. The freeze-drying process is preferably carried out over a space of time of about 15 to 72 hours. Following the freeze-drying process, the composition according to the invention has a residual water content of less than 10%, more preferably less than 5%, still more preferably less than 1%. The freeze-dried compositions or molded articles can be subjected to a further subsequent treatment, such as laminating, cutting, punching or stamping or the like.

Both the releasing agents and the auxiliary substances are directly contained in the composition according to the invention, together with the stabilized active substance, and all substance groups are homogeneously distributed in the composition or the carrier material, without a need for keeping them stable and easily available in the composition by an additional effort with respect to their separation. Thus, the composition according to the invention is particularly suitable as a product for external application, particularly as a cosmetic product, with its use as a skin care product being particularly preferred. What is also conceivable is the use of the composition according to the invention as a therapeutic product, in particular for external use.

In this case, external application is carried out by the composition according to the invention being wetted with or dissolved in an aqueous phase, preferably with water or an aqueous solution, which may optionally contain other active ingredients and/or auxiliary substances. Depending on the amount of liquid and the solubility of the carrier materials used, the composition can be completely dissolved while a solution is being formed, it can dissociate while a gel is formed, or, in the case of cross-linked and insoluble embodiments, it can swell while its shape is being maintained. If the composition according to the invention is dissolved in a larger amount of water, this is, as a rule, a bathing application, and, according to the invention, this application is included in the external application. Preferably, application is carried out by the composition being wetted directly on the skin, e.g. on the directly application location or on the palm of the hand, with a small amount of water or of a solution of active ingredients and/or of auxiliary substances, while a solution, a gel or a swollen matrix or layer is formed, and by then being applied either there or from there onto the area of the body to be treated, by being rubbed on, massaged in, applied onto it or laid onto it.

Furthermore, the present invention also relates to a combination comprising at least one of the compositions according to the invention as well as at least one aqueous solution, which optionally contains one or more other active ingredients and/or auxiliary substances, in a spatial arrangement that belongs together (application pack, set, kit of parts, etc.).

The external application can be carried out by the end consumers themselves, or within the context of professional treatment concept, e.g. in wellness/cosmetic treatments and/or in therapeutic treatments.

### EXAMPLES

### Example 1

Rapidly soluble composition of polysaccharide carrier with vitamin C derivatives and an enzyme as a releasing agent.

### Manufacture of the Composition:

2.0 g Sodium alginates (Satialgine US 3001)

0.2 g Ascorbyl glucoside

10 g Glucoamylase (Novozym 300 GL; 10-40%)

87.8 g RO water (desalted water, reverse osmosis)

The alginate powder is added to the RO water by means of a mixer until there is a homogeneous mixture. Then, the ascorbyl glucoside is mixed in. Optionally, the suspension is then adjusted to a pH value of 4 to 5 using hydrochloric acid, and cooled to <4° C. Within <8 seconds, the enzyme solution (glucoamylase) is admixed into the cooled suspension. The suspension is then poured into molds, frozen at temperatures <−10° C., separated from the molds and then subjected to freeze drying.

### Example 2

Rapidly soluble composition of polysaccharide carrier with auxiliary substances, vitamin C derivative and an enzyme as a releasing agent.

### Manufacture of the Composition:

2.0 g Sodium alginates (Satialgine US 3001)

0.2 g Ascorbyl glucoside

4.0 g Capryl/capric acid triglycerides

10 g Glucoamylase (Novozym 300 GL; 10-40%)

85.8 g RO water (desalted water, reverse osmosis)

The alginate powder is added to the RO water by means of a mixer until there is a homogeneous mixture. Then, the ascorbyl glucoside and the triglycerides are mixed in. Optionally, the suspension is then adjusted to a pH value of 4 to 5 using hydrochloric acid, and cooled to <4° C. Within <8 seconds, the enzyme solution (glucoamylase) is admixed into the cooled suspension.

The suspension is then poured into molds, frozen at temperatures <−10° C., separated from the molds and then subjected to freeze drying.

### Example 3

Rehydratable, gel-forming composition of polysaccharide carrier with auxiliary substances, vitamin C derivative and an enzyme as a releasing agent.

### Manufacture of the Composition:

1.7 g Sodium alginates (Satialgine US 3001)

0.4 g Sodium carboxymethylcellulose

0.2 g Ascorbyl glucoside

0.4 g Squalane

0.1 g PPG-15 stearyl ether

0.1 g PEG-40 sorbitan peroleates

0.2 g Viscose fibers (rayon)

10 g Glucoamylase (Novozym 300 GL; 10-40%)

86.9 g RO water (desalted water, reverse osmosis)

The alginate powder and the carboxymethylcellulose are added to the RO water by means of a mixer until there is a homogeneous mixture. Then, the ascorbyl glucoside and the auxiliary substances squalane, PPG-15 stearyl ether and PEG-40 sorbitan peroleate are stirred in, and the rayon fibers are subsequently mixed in a homogenous manner. Optionally, the suspension is then adjusted to a pH value of 4 to 5 using hydrochloric acid, and cooled to <4° C. Within <8 seconds, the enzyme solution (glucoamylase) is admixed into the cooled suspension.

The suspension is then poured into molds, frozen at temperatures <−10° C., separated from the molds and then subjected to freeze drying.

Optionally, the freeze-dried compositions thus obtained are subjected to a subsequent mechanical treatment, such as laminating, cutting, punching, packaging or the like.

### Example 4

### Use of the Compositions from Example 1-2

Immediately, prior to topical application, the freeze-dried compositions from the examples 1 and 2 are wetted with, or dissolved in, an aqueous phase, preferably water, thermal water or a solution of active ingredients, which may optionally contain other active ingredients and/or auxiliary substances. This can take place either in a suitable container or in the palm of the hand. After wetting the composition, the solution or gel forming is applied onto the area to be treated, and is rubbed or massaged in at that location.

### Example 5

### Use of the Compositions from Example 3

Immediately prior to topical application, the freeze-dried compositions from examples 3 are wetted with, or dissolved in, an aqueous phase, preferably water, thermal water or a solution of active ingredients, which may optionally contain other active ingredients and/or auxiliary substances. This can take place either in a suitable container or in the palm of the hand, or preferably directly on the desired area of topical application. After wetting the composition, the solution or gel forming is optionally applied onto the area to be treated, and is rubbed or massaged in at that location.

1. Composition comprising at least one carrier material and at least one active ingredient present in stabilized form, and at least one agent for forming an active ingredient from the stabilized form upon addition of an aqueous phase to the composition, wherein the composition is obtained by freeze drying.

2. Composition according to claim 1, wherein the carrier material is hydrophilic.

3. Composition according to claim 1, wherein the carrier material comprises at least one hydrocolloid.

4. Composition according to claim 1, wherein the carrier material is selected from the group consisting of: polysaccharides, glucosaminoglycanes, proteins and synthetic polymers.

5. Composition according to claim 1, wherein the stabilized active ingredient is a precursor of an active ingredient and/or a derivative of an active ingredient.

6. Composition according to claim 1, wherein the stabilized active ingredient is selected from the group consisting of: vitamins, fragrant substances, aromas, pharmacological active ingredients and cosmetic active ingredients.

7. Composition according to claim 1, wherein the stabilized active ingredient is a vitamin derivative.

8. Composition according to claim 1, wherein the agent for forming the active ingredient is selected from the group consisting of: catalysts, acids, bases and redox agents.

9. Composition according to claim 1, wherein the agent for forming the active ingredient is selected from enzymes.

10. Composition according to claim 1, comprising at least one auxiliary substance.

11. Composition according to claim 1, in which a homogeneous distribution of the ingredients is present in a matrix of the carrier material.

12. Composition according to claim 1, comprising:
at least 10% by wt. of carrier materials,
at least 0.01% by wt. of stabilized active ingredients,
at least 0.0001% by wt. of agents for forming the active ingredient upon addition of an aqueous phase,
0 to 80% by wt. of one or more auxiliary substances, and
less than 10% by wt. of water,
relative to the total quantity of the composition.

13. Composition according to claim 1, obtainable by a process comprising the steps of:
a) preparing an aqueous solution or suspension comprising at least one carrier material, at least one stabilized active ingredient and at least one agent for forming the active ingredient and, optionally, one or more auxiliary substances,
b) pouring the solution or suspension into a mold,
c) freezing the solution or suspension in the mold and
d) freeze drying the frozen solution or suspension.

14. Composition according to claim 1, which is present as a molded article.

15-18. (canceled)

19. Composition comprising at least one carrier material and at least one cosmetic or pharmaceutical active ingredient present in stabilized form, and at least one agent for forming an active ingredient from the stabilized form upon addition of an aqueous phase to the composition.

20. A method of using the composition according to claim 1, the method comprising wetting the composition with an aqueous phase and applying the wetted composition externally.

21. The method according to claim 20 wherein the wetted composition is applied externally onto the skin or the hair.

22. The method of using the composition according to claim 20 wherein the active ingredient is a cosmetic agent.

23. The method of using the composition according to claim 20 wherein the active ingredient is a pharmaceutical agent.
